(19) United States
(12) Patent Application Publication
(10) Pub. No.: US 2013/0239663 A1
(43) Pub. Date: Sep. 19, 2013

(54) APPARATUS FOR EVALUATING LUBRICAN

(71) Applicant: HITACHI HIGH-TECHNOLOGIES CORPORATION, Tokyo (JP)

(72) Inventors: Yasuo YAHAGI, Tokyo (JP); Masayuki KOBAYASHI, Fujisawa (JP); Hiroaki MITO, OR (US); Tomohiro KUDO, Hitachinaka (JP); Tomonori SAEKI, Yokosuka (JP)

(73) Assignee: Hitachi High-Technologies Corporation, Tokyo (JP)

(21) Appl. No.: 13/872,950

(22) Filed: Apr. 29, 2013

Related U.S. Application Data
(62) Division of application No. 11/964,754, filed on Dec. 27, 2007.

(30) Foreign Application Priority Data
Dec. 27, 2006 (JP) 2006-352761

(51) Int. Cl. G01N 33/00

(52) U.S. Cl. CPC G01N 33/00
USPC 73/53.05

(57) ABSTRACT

An apparatus having a vacuum vessel that has a mechanism using a lubricant therein and not causing defects and faults to samples introduced into the vacuum vessel even it is an apparatus where lubricating oil or grease is applied is provided. An apparatus having a vacuum vessel that has a mechanism using a lubricant therein such as CD-SEM, in which a lubricant (oil, grease) whose adsorption amount per minute to a surface of a material introduced into the vacuum vessel of an apparatus for evaluating a lubricant after the start of vacuum evacuation and after reaches a quasi-equilibrium state is below 0.09 ng/cm² is employed.

### CROSS-REFERENCE TO RELATED APPLICATION

The present application claims priority from Japanese Patent Application No. JP 2006-352761 filed on Dec. 27, 2006, the content of which is hereby incorporated by reference into this application.

This application is a Divisional application of application Ser. No. 11/964,754, filed Dec. 27, 2007, the contents of which are incorporated herein by reference in their entirety.

### TECHNICAL FIELD OF THE INVENTION

The present invention relates to an apparatus having a vacuum vessel. More particularly, the present invention relates to a technique effectively applied to a vacuum apparatus such as a semiconductor manufacturing apparatus having a vacuum vessel.

### BACKGROUND OF THE INVENTION

In recent years, miniaturization and highly integrated configurations of semiconductor devices have been remarkably progressed, and in particular, CMOS (Complementary MOS) semiconductor devices combining n-type and p-type MOSs are superior to bipolar type devices in view of their power consumption and ultrafine and highly integrated configuration, and have been developed day after day. At the most advanced research and development level made public in the IEDM (International Electron Devices Meeting) in December, 2004, the gate length of the planar type CMOS has reached to 5 nm, i.e., the size of about 18 Si atoms (H. Wakabayashi, T. Ezaki, M. Hane, T. Ikezawa, T. Sakamoto, H. Kawaura, S. Yamagami, N. Ikarashi, K. Takeuchi, T. Yamamoto, and T. Mogami, “Transport properties of sub-10-nm planar-bulk-CMOS devices,” in International Electron Device Meeting Tech. Dig., San Francisco, Calif., Dec. 13-15, 2004, pp. 429-432, (2004) (Non-Patent Document 1)).

At mass production level, the production of 90-nm process generation has started in 2004, and at the moment of the present application, 65-nm process generation is about to begin. In order to shift to the mass production in a short period while securing ensuring reliability from the product development, it is indispensable to shorten and optimize conditions of mass production process. To gain high reliability, it is primarily required to limit the designed geometric structure certainly in an acceptable dimension range. For this purpose, the dimension measurement of ultrafine patterns formed by lithography technology is indispensable. For this measurement, as an inline inspecting apparatus, electron microscope CD-SEM (Critical-Dimension Scanning Electron Microscope) designed and manufactured exclusively for dimension measurement, that is, a length measuring SEM is employed, and from the viewpoints of securement of semiconductor device reliability and the semiconductor manufacturing period, a highly precise and high throughput measurement is required.

Further, on the other hand, for improving the mass production efficiency, the Si wafer size has been made larger to 8 inches, 12 inches in diameter (so-called 300 mm wafer). Therefore, in addition to the large size of a sample chamber as the vacuum vessel for CD-SEM, high precision and high speed drive of the sample stage are indispensable. Since the mechanical drive system where the sample stage is put on a guide rail and driven at a high speed by a ball screw is employed, lubricating oil is applied to the guide rail and the ball screw. As the lubricating oil, oil of low vapor pressure used widely as vacuum oil is used. Further, lubricating oil and grease are applied to other moving parts in the vacuum vessel in the same manner. Meanwhile, recently, conspicuous defects and faults considered to be caused by the lubricating oil where components included in oil attach onto the wafer surface and pose contamination to the wafer have become obvious in the lithography process of semiconductors.

For example, as suggested in the paragraphs [0122] to [0125] in Japanese Patent Application Laid-Open Publication No. 2002-250707 (Patent Document 1), some problems such as organic contamination supposed to arise from oil and the like have occurred. And, it is considered that according to the present invention, such contamination is solved by removing it by plasma.

In the vacuum apparatus having moving parts in the vacuum vessel, lubricating oil and grease are applied in the same manner as in the above CD-SEM, and in the case when such an apparatus is used, conspicuous defects and faults considered to arise from components included in oil have occurred to samples.

### SUMMARY OF THE INVENTION

Meanwhile, in the above-said technology of semiconductor device, as well as the ultrafine and highly integrated configuration of semiconductor devices, the development cycle of products has become shorter and the prices of mass production products have become lower. In order to secure reliability from the product development and shift to the mass production in a short period, it is indispensable to shorten and optimize conditions of mass production process. The mask and wafer for lithography used in the manufacturing process must not have any defect. In order to obtain highly reliable products, it is primarily required to contain a designed geometric structure precisely in an allowable dimension range. For this purpose, the dimension measurement of ultrafine patterns formed by lithography technology is indispensable. For this measurement, as an inline inspecting apparatus, an electron microscope CD-SEM designed and manufactured exclusively for dimension measurement is used, and from the viewpoints of securement of semiconductor device reliability and the semiconductor manufacturing period, a high-precision and high-throughput measurement is required.

Therefore, in addition to enlarging the size of a sample chamber as a vacuum vessel for CD-SEM, high-precision and high-speed drive of a sample stage is indispensable. Since a mechanical drive system where the sample stage is put on a guide rail and driven at a high speed by a ball screw is employed, lubricating oil is applied to the guide rail and the ball screw. As the lubricating oil, a lubricating oil of low vapor pressure used widely as vacuum oil is used. On the other hand, the manufacturing processes of products are complicated, and despite that the oil having a low vapor pressure used widely in the vacuum vessel is used, conspicuous defects and faults considered to be caused by contamination of the lubricating oil components, more particularly, components included in the lubricating oil attaching onto the wafer surface pose contamination of the wafer surface and it has become apparent in the lithography process of semiconductor.

With regard to the contamination attaching to the wafer surface, the inventors of the present invention have conducted various physical and chemical analyses, and as the result of detailed examination of the measurement results, it has been found that the contamination is caused by the components of lubricating oil attaching onto the wafer surface. The lubricating oil used in the vacuum vessel is lubricating oil of low vapor pressure, but even in vacuum, an extremely small amount of lubricating oil components will be vaporized, and then attach on the wafer surface as contamination. And as a consequence, the problems where it causes conspicuous defects and faults in the semiconductor process have become apparent.

Accordingly, an object of the present invention is to provide an apparatus that has a vacuum vessel including a mechanism using a lubricant inside and does not cause defects and faults to samples introduced into the vacuum vessel even in an apparatus where lubricating oil and grease are applied.

The above and other objects and novel characteristics of the present invention will be apparent from the description of this specification and the accompanying drawings.

The typical ones of the inventions disclosed in this application will be briefly described as follows.

In order to achieve the above object, the present invention is an apparatus having a vacuum vessel that includes a mechanism using a lubricant therein, in which a lubricant whose adsorption amount per minute to a surface of a material introduced into the vacuum vessel of an apparatus for evaluating a lubricant is less than 0.09 ng/cm² after reaching a quasi-equilibrium state after starting vacuum evacuation is employed in the mechanism of the vacuum vessel.

The effects obtained by typical aspects of the present invention will be briefly described below.

According to the present invention, in the apparatus having a vacuum vessel that includes a mechanism using a lubricant therein, even it is an apparatus in which lubricants are applied, it is possible to control the occurrence of defects and faults including contamination to samples in processes after introduction into the vacuum vessel.

### BRIEF DESCRIPTIONS OF THE DRAWINGS

FIG. 1 is a diagram showing an example of a CD-SEM having a mechanism using lubricants in a vacuum vessel thereof according to an embodiment of the present invention;

FIG. 2 is a diagram showing an example of a stage system in the CD-SEM according to the embodiment of the present invention;

FIG. 3A is a diagram showing an example of an apparatus for evaluating a lubricant and it is a top view of the vacuum vessel unit thereof according to the embodiment of the present invention;

FIG. 3B is a diagram showing an example of an apparatus for evaluating a lubricant and it is a front view of the vacuum vessel unit thereof according to the embodiment of the present invention;

FIG. 4 is a diagram showing an example of a temperature profile for performing a thermal cleaning of the vacuum vessel of the apparatus for evaluating a lubricant according to the embodiment of the present invention;

FIG. 5A is a front view showing an example of the shape of a stainless steel plate for applying lubricating oil used in the apparatus for evaluating a lubricant according to the embodiment of the present invention;

FIG. 5B is a side view showing an example of the shape of the stainless steel plate for applying lubricating oil used in the apparatus for evaluating a lubricant according to the embodiment of the present invention;

FIG. 6 is a diagram showing an example of a measurement result by a quadrupole mass spectrometer in the case when lubricating oil is not used evaluated by use of the apparatus for evaluating a lubricant according to the embodiment of the present invention;

FIG. 7 is a diagram showing an example of a measurement result by a crystal oscillator in the case when lubricating oil is not used evaluated by use of the apparatus for evaluating a lubricant according to the embodiment of the present invention;

FIG. 8 is a diagram showing an example of measurement results by a quadrupole mass spectrometer in the case when three kinds of lubricating oils and the case when lubricating oil is not used evaluated by use of the apparatus for evaluating a lubricant according to the embodiment of the present invention;

FIG. 9 is a diagram showing an example of measurement results by a crystal oscillator in the case when three kinds of lubricating oils and the case when a lubricating oil is not used evaluated by use of the apparatus for evaluating a lubricant according to the embodiment of the present invention; and

FIG. 10 is a diagram showing examples of: an average adsorption amount per minute obtained by linearly fitting the measurement results by the crystal oscillator in the cases when three kinds of lubricating oils are used and in the case when lubricating oil is not used by use of the apparatus for evaluating a lubricant according to the embodiment of the present invention; the number of defects and faults in a lithography process; and measurement results of contact angle of purified water.

### DESCRIPTIONS OF THE PREFERRED EMBODIMENTS

Hereinafter, embodiments of the present invention will be described in detail with reference to the accompanying drawings. Note that components having the same function are denoted by the same reference symbols throughout the drawings for describing the embodiment, and the repetitive description thereof will be omitted.

### OUTLINE OF THE EMBODIMENTS

In order to shift to the mass production of semiconductor devices in a short period while securing reliability from the product development, it is indispensable to shorten and optimize conditions of mass production process. To obtain high reliability, it is primarily required to contain a designed geometric structure precisely in an allowable dimension range. For this purpose, a dimension measurement of ultrafine patterns formed by lithography technology is indispensable. As an inline inspecting apparatus of the semiconductor process, an electron microscope CD-SEM designed and manufactured exclusively for dimension measurement is employed, and from the viewpoints of securement of semiconductor device reliability and the semiconductor manufacturing period, a high-precision and high-throughput measurement is required.

Therefore, in addition to enlarging the size of a sample chamber as a vacuum vessel for CD-SEM, high-precision and high-speed drive of a sample stage is indispensable. Since a mechanical drive system where the sample stage is put on a guide rail and driven at a high speed by a ball screw is employed, lubricating oil is applied to the guide rail and the ball screw. As the lubricating oil, a lubricating oil of low vapor pressure used widely as vacuum oil is used. On the other hand, the manufacturing processes of products are complicated, and despite that the oil having a low vapor pressure used widely in the vacuum vessel is used, conspicuous defects and faults considered to be caused by contamination of the lubricating oil components, more particularly, components included in the lubricating oil attaching onto the surface of a wafer (also described as “sample”) pose contamination of the wafer surface and it has become apparent in the lithography process of semiconductors.

The contamination by lubricating oil components to a wafer inspected by CD-SEM occurs when the vacuum vessel is pressure-reduced in a situation where the wafer and the lubricating oil coexist in the vacuum vessel of CD-SEM. Therefore, the inventors of the present invention have devised a method for preliminarily investigating vaporization/adsorption behaviors in vacuum of the lubricating oil to be used in the vacuum vessel. A feature of the present invention is an apparatus for evaluating a lubricant for performing the measurement evaluation of vaporization/adsorption behaviors of the lubricating oil for realizing the method.

Hereinafter, first, CD-SEM as an inline inspecting apparatus of semiconductor process is described, and further, the apparatus for evaluating a lubricant for performing the measurement evaluation of vaporization/adsorption behaviors of the lubricating oil is described.

(CD-SEM)

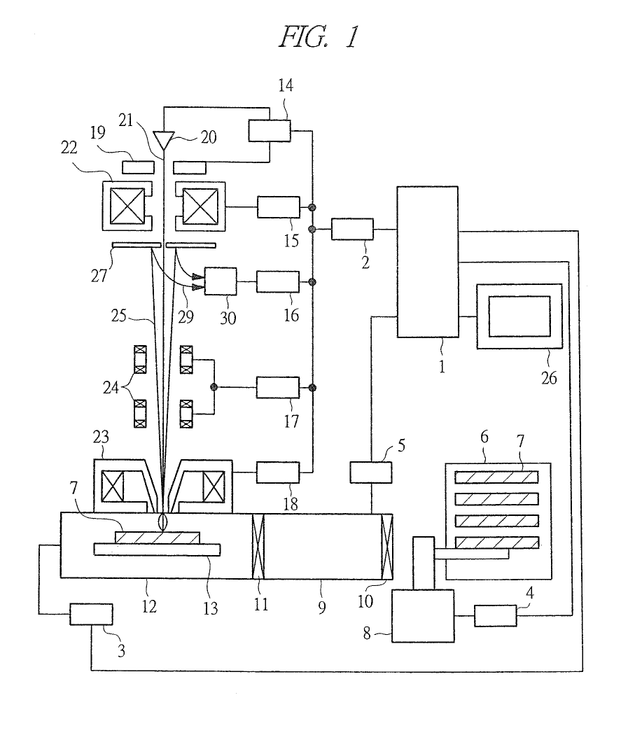

With reference to FIG. 1, an example of a CD-SEM according to an embodiment of the present invention is described. The CD-SEM comprises a controller 1, an electron-optical system controller 2 that is controlled by the controller 1, a stage controller 3, a sample transfer controller 4, and a sample exchange chamber controller 5, and further an electron optical system, a stage system, a sample transfer system, and a sample exchange chamber that are controlled by these controllers 2, 3, 4 and 5.

In this CD-SEM, the controller 1 controls the electron-optical system controller 2, the stage controller 3, the sample transfer controller 4, and the sample exchange chamber controller on the basis of acceleration voltage, sample information, measurement position information, wafer cassette information and the like inputted by an operator via user interface that is not shown.

The sample transfer controller 4 received a command from the controller 1 controls a transfer robot 8 so that an arbitral wafer 7 is moved from a wafer cassette 6 to a predefined position in a sample exchange chamber 9. The sample exchange chamber controller 5, in sync with the movement of the wafer 7 in and out of the sample exchange chamber 9, performs control so as to open and close gate valves 10 and 11. Further, the sample exchange chamber controller 5 controls a vacuum pump (not shown) that evacuates the inside of the sample exchange chamber 9 to vacuum, and when the gate valve 11 opens, it forms a similar vacuum as that of a sample chamber 12 in the inside of the sample exchange chamber 9. The wafer 7 that enters in the sample exchange chamber 9 is sent to the sample chamber 12 via the gate valve 11 and fixed onto a sample stage 13.

The electron-optical system controller 2 controls a high voltage controller 14, a condenser lens controller 15, an amplifier 16, a deflection signal controller 17, and an objective lens controller 18 in accordance with the command from the controller 1.

By an extraction electrode 19, an electron beam 21 extracted from an electron source 20 is focused by a condenser lens 22 and an objective lens 23 and radiated onto the wafer arranged on the sample stage 13. The electron beam 21 is made to scan on the wafer 7 one-dimensionally or two-dimensionally by a deflector 24 that received signals from the deflection signal controller 17.

Due to the radiation of the electron beam 21 onto the wafer 7, a secondary charged particle 25 discharged from the wafer 7 is converted into secondary electrons 29 by a secondary electron conversion electrode 27, and the secondary electrons 29 are trapped by a secondary charged particle detector 30, and used as a luminance signal of a display screen of a display device 26 via the amplifier 16.

Further, by synchronizing deflection signals of the display device 26 and deflection signals of the deflector 24, it is possible to reproduce a pattern shape on the wafer on the display device 26.

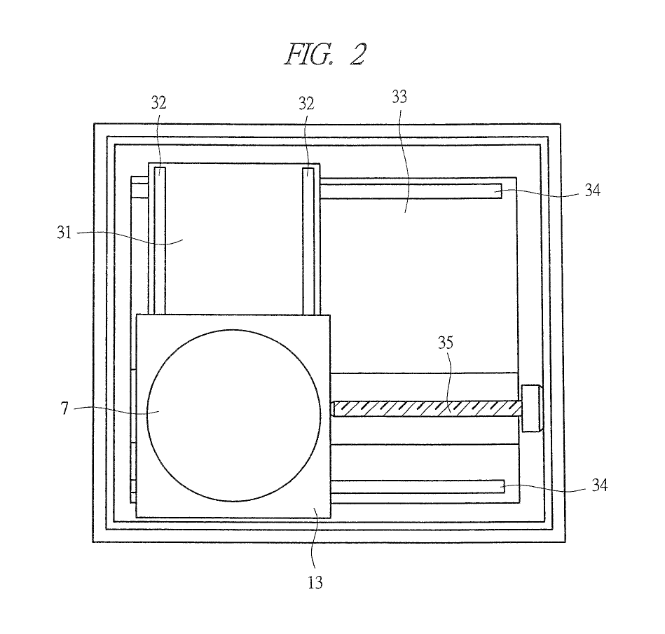

Next, with reference to FIG. 2, an example of the stage system is described. On a guide rail 34 and its base 33, a guide rail 32 and its base 31 are arranged, and further thereon, the sample stage 13 is arranged. The wafer 7 is fixed onto the sample stage 13. The rotation movement of a ball screw 35 is converted into linear movement and the guide rail 32 and its base 31, the sample stage 13, and the wafer 7 can move on the guide rail 34 linearly (in X direction). In the same manner, the rotation movement of a ball screw (not shown) is converted into linear movement, and the sample stage 13 can move on the guide rail 34 linearly (in Y direction).

In such a CD-SEM, as mechanisms to use a lubricant in the sample chamber 12 as a vacuum vessel, there are the guide rails 32 and 34, the ball screw 35 and the like, and lubricating oil is applied to these guide rails 32 and 34, the ball screw 35 and the like.

(Apparatus for Evaluating a Lubricant)

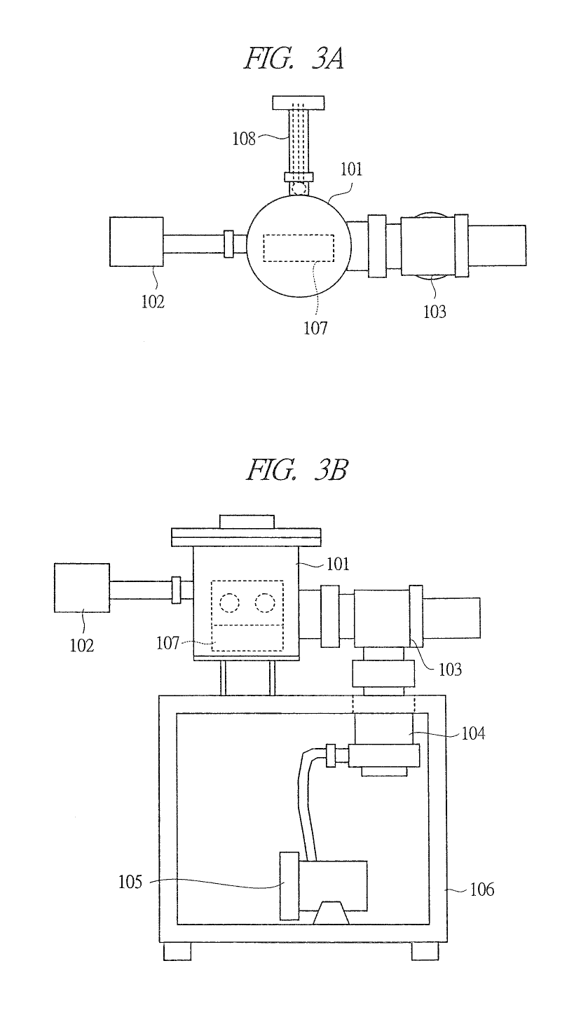

With reference to FIG. 3A (top view of vacuum vessel portion) and FIG. 3B (front view of vacuum vessel portion), an example of the apparatus for evaluating a lubricant is described. The apparatus for evaluating a lubricant comprises a stainless-steel vacuum vessel 101, a quadrupole mass spectrometer 102 connected to one side of the stainless-steel vacuum vessel 101, an angle type valve 103 connected to the other side of stainless-steel vacuum vessel (also simply referred to as vacuum vessel) 101, a turbo-molecular pump 104 connected to the angle type valve 103, a dry scroll pump 105 connected to the turbo-molecular pump 104, a counter 106 of the apparatus for evaluating a lubricant and the like.

This apparatus for evaluating a lubricant is an apparatus where a stainless steel plate for lubricant application 107 is put in the stainless steel vacuum vessel 101, and the measurement of vaporized/adsorbed components from the lubricant can be carried out by a crystal oscillator element 108 attached to one of flanges of the vacuum vessel 101.

The size of the main portion of the stainless-steel vacuum vessel 101 is approximately 25 cm in diameter and 30 cm in height. To this, via a copper gasket of ICF152 standard flange and the angle type valve 103, the vacuum vessel 101 can be evacuated by use of the turbo-molecular pump 104 whose throughput is 150 L per minute, and the dry scroll pump 105 whose throughput is 250 L per minute as auxiliary evacuation. Further, although not shown in FIG. 3A and FIG. 3B, a B-A gauge and a Pirani gauge are attached as vacuum gauges. Furthermore, although not shown in FIG. 3A and FIG. 3B either, high purity nitrogen gas can be introduced into the vacuum vessel 101 when the vacuum is released.

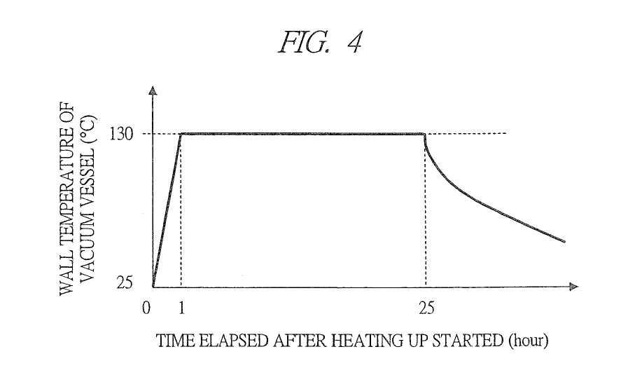

The crystal oscillator element 108 and the quadrupole mass spectrometer 102 were respectively attached beforehand to ports having flanges of ICF70 standard of the stainless steel vacuum vessel 101 via the copper gasket and a nipple having a length of about 20 cm. As the crystal oscillator element 108, TM-400 made by MAXTEK Inc. was employed, and as the quadrupole mass spectrometer 102, SPECTRA (Trademark) made by MICROVISION was employed. While the vacuum vessel 101 was evacuated, the entire vacuum vessel 101 was heated up by, for example, a temperature profile as shown in FIG. 4, and heated up to 130° C. for 24 hours. In this manner, the vacuum vessel 101 was cleaned.

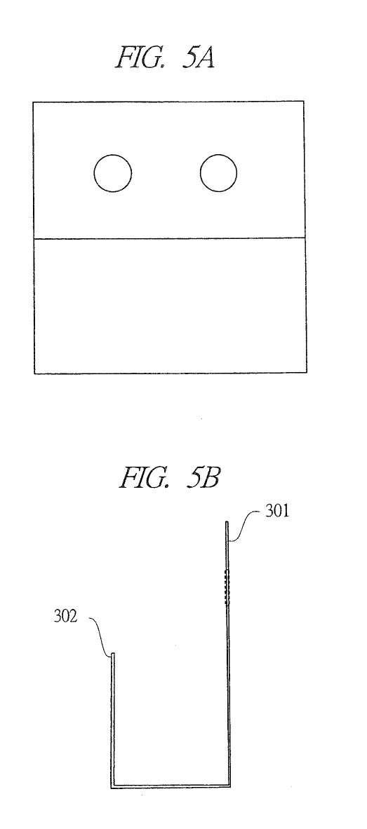

While the vacuum vessel 101 was evacuated, when the temperature of the vacuum vessel 101 was lowered to around 70° C., degassing operation of a head of the quadrupole mass spectrometer 102 was carried out for about 5 minutes, and thereafter, the vacuum vessel 101 was continued to be evacuated until the temperature of the vacuum vessel 101 reached around the room temperature. At this moment, the vacuum pressure reached nearly 5×10⁻⁵ Pa. Thereafter, water at about 25° C. was made to flow via a temperature adjuster to a water-cooling pipe of the crystal oscillator element 108, and this operation was carried out over 2 hours. A basic resonance frequency of the crystal oscillator element 108 was 6.04 MHz, and about 20 nm of chrome (Cr) and about 155 nm of gold (Au) formed in a pattern on an AT-cut quartz (crystal) crystal plate and are made into electrodes. The stainless steel plate for lubricant application 107 was made of a plate of size 31 cm×16 cm, thickness 1.5 mm bent and processed so as to comprise 3 parts as shown in FIG. 5A (front view) and FIG. 5B (side view).

By making the shape of the plate to which the lubricant is applied constant, it is possible to make the application amount of the lubricant nearly constant, and it is possible to quantitatively compare characteristics of different lubricating oils. At the longer fold-back side, that is, at the side of 301 in FIG. 5, two circular holes having a diameter of 2 cm are made for easily operating application of the lubricating oil to the plate and arranging the plate into the vacuum vessel. Hereinafter, this stainless-steel plate for lubricant application 107 is referred to as test plate for lubricant application.

Meanwhile, the vacuum vessel and the turbo pump in the above evaluation apparatus are simply examples, and they are not limited by the size of the vacuum vessel and the throughput of the pump as long as the same vacuum performance can be obtained.

By use of the above-described apparatus for evaluating a lubricant for performing the measurement evaluation of vaporization/adsorption components of the lubricant, evaluation of the lubricant was carried out. As an example, three kinds of fluorinated lubricants, namely, a lubricating oil A, a lubricating oil B, and a lubricating oil C processed for lowering the vapor pressure were evaluated. The procedures are described hereinafter.

First, the vacuum vessel 101 and the measuring instruments were cleaned and the measuring instruments were stabilized. While the vacuum vessel 101 was evacuated, the entire vacuum vessel 101 was heated up by the temperature profile as shown in FIG. 4, and heated at 130° C. for 24 hours. Accordingly, the vacuum vessel 101 was cleaned. While the vacuum vessel 101 was evacuated, when the temperature of the vacuum vessel 101 lowered to around 70° C., degassing operation of the head of the quadrupole mass spectrometer 102 was carried out for about 5 minutes. Thereafter, the vacuum vessel 101 was continued to be evacuated until the temperature of the vacuum vessel 101 reached around the room temperature. According to the foregoing, the measuring instruments were cleaned. Further thereafter, water at about 25° C. was made to flow via the temperature adjuster to the water cooling pipe of the crystal oscillator element 108, and this operation was carried out over 2 hours. According to the foregoing, the cleaning of the vacuum vessel 101 and the measuring instruments and the stabilization of the measuring instruments were completed.

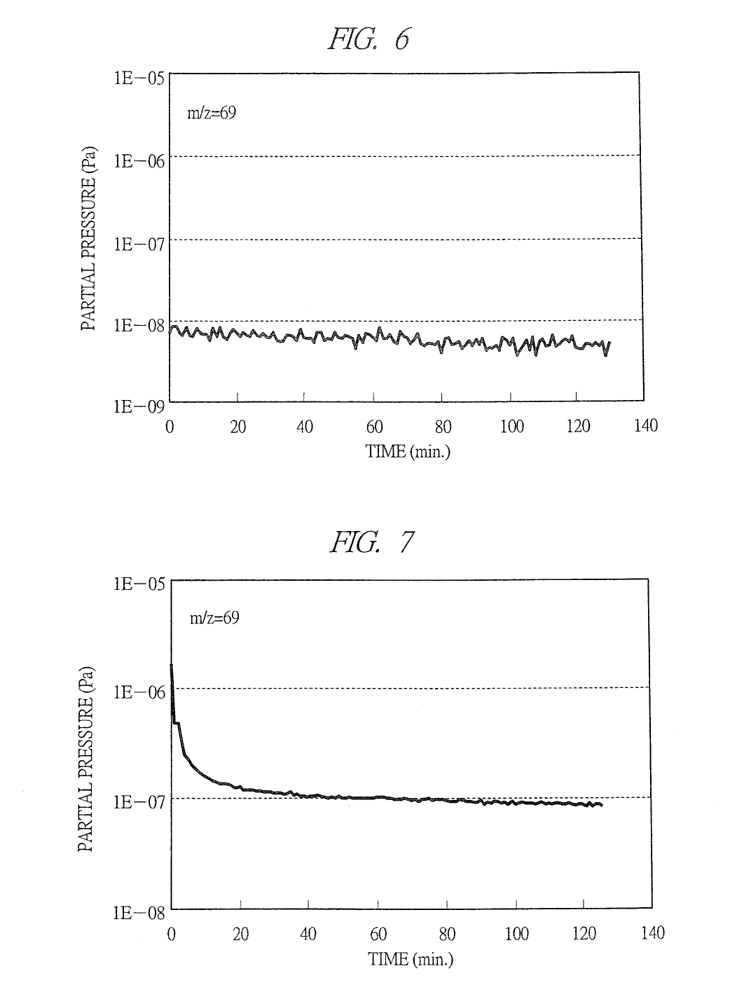

Next, in order to acquire background data of the apparatus system, while lubricating oil was not applied to the test plate for lubricant application (107) shown in FIG. 5, the stainless-steel plate was placed in the vacuum vessel 101, and after the vacuum vessel 101 was closed with a lid, the vacuum vessel 101 was evacuated by vacuum pumps (the turbo-molecular pump 104, the dry scroll pump 105). From when 2 to 3 minutes has elapsed after the evacuation started, the time changes of partial pressure were acquired by the quadrupole mass spectrometer 102. As an example, the time changes concerning a ratio of a mass number m and the number of charges z, which is m/z=69 is shown in FIG. 6.

In the case when lubricating oil is not applied to the test plate for lubricant application, although the partial pressure slightly decreases with lapse of the evacuation time, it may be regarded as nearly constant.

Next, the lubricating oil A was applied to the test plate for lubricant application, and in the same manner, the time changes of partial pressure were acquired by the quadrupole mass spectrometer 102, and the result is shown in FIG. 7.

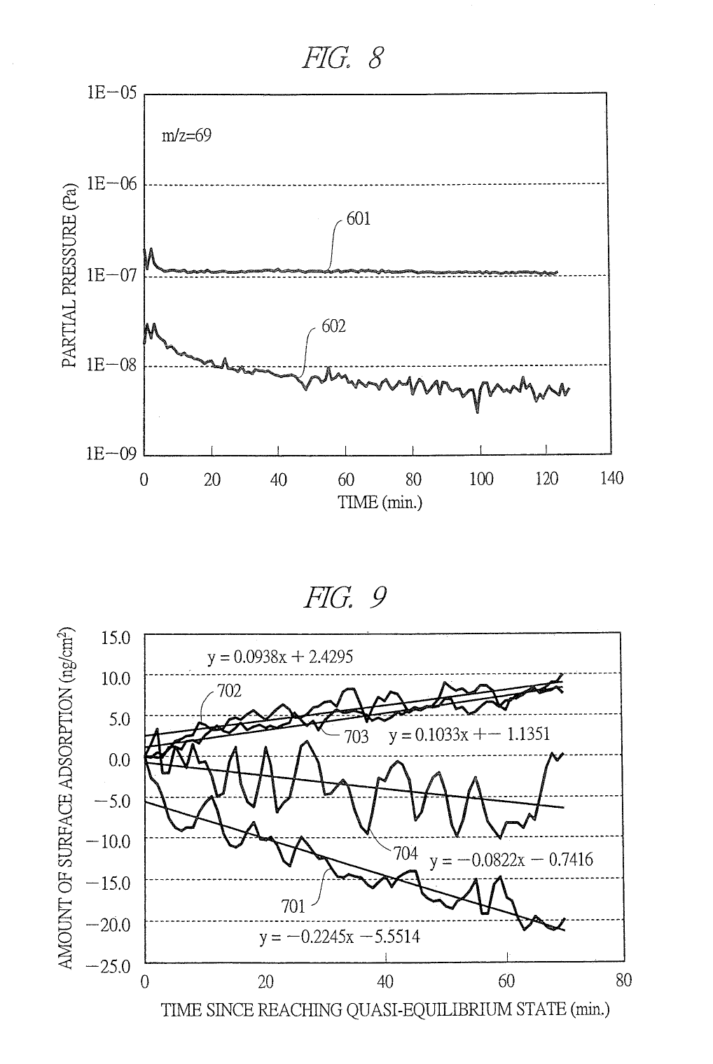

And further, the cleaning of the vacuum vessel 101 was carried out, and anther lubricating oil B was applied to the cleaned test plate for lubricant application, and the plate was placed in the vacuum vessel 101. And after the vacuum vessel 101 was closed with the lid, the vacuum vessel 101 was evacuated by the vacuum pumps. After the time change of partial pressure was acquired by the quadrupole mass spectrometer 102, the cleaning and the like of the vacuum vessel 101 were carried out in the same manner, and further, with regard to the other lubricating oil C, the time change of partial pressure were acquired by the quadrupole mass spectrometer 102 in the same manner. These results are shown in FIG. 8. In FIG. 8, 601 shows the time change of partial pressure concerning m/z=69 of the lubricating oil B, and 602 shows that of the lubricating oil C.

Referring to the time changes of partial pressure acquired by the quadrupole mass spectrometer 102 shown in FIG. 7 and FIG. 8, it is found that, in the respective cases, the partial pressure decreases rapidly until about 20 minutes have elapsed after the evacuation started, then, the decrease amount per unit time, i.e., the decrease ratio declines gradually, and it becomes nearly constant after about 30 minutes from the evacuation started. The system state where the ratio of the partial pressure decrease observed by use of the quadrupole mass spectrometer 102 becomes extremely low and the partial pressure decrease can be regarded to be substantially constant as seen in the time period of about several hours is defined as “quasi-equilibrium state” herein. After reaching to the quasi-equilibrium state judged by the time changes of partial pressure measured by the quadrupole mass spectrometer 102, the time changes of the resonance frequency of the crystal oscillator element 108 were observed. Here, it is shown that the changes of the resonance frequency of the crystal oscillator element 108 can be converted into the weight area density of materials that attached onto the surface of the crystal oscillator element, according to the following Equation 1 taught in Sauerbrey (G. Sauerbrey, “Verwendung von Schwingquarzen zur Wägung dünner Schichten and zur Mikrowägung,” Zeitschrift für Physik, 155, pp. 206-222 (1959) (Non-Patent Document 2)—p 208).

Δm/F=−ρ_{Q}·d·Δf/f Equation 1

Here, Δm means the amount of weight increase of materials attaching onto the surface of the crystal oscillator element, F means an area of the surface of the crystal oscillator element where the materials attach thereon, ρ_{Q} means a density of the crystal oscillator crystal, d means an thickness of the crystal oscillator crystal, ΔF means an change of the resonance frequency of the crystal oscillator corresponding to the amount of weight increase of the material attaching onto the surface, and f means a basic resonance frequency of the crystal oscillator. Results of the observation on the time changes of the resonance frequency of the crystal oscillator in four cases that lubricating oils A, B and C are applied to the test plate for lubricant applications respectively and nothing is applied to the test plate for lubricant application are shown in FIG. 9. In FIG. 9, 701 shows the result of the time changes in the case when nothing is applied, 702 shows the result in the case when the lubricating oil A is applied, 703 shows the result in the case when the lubricating oil B is applied, and 704 shows the result in the case when the lubricating oil C is applied.

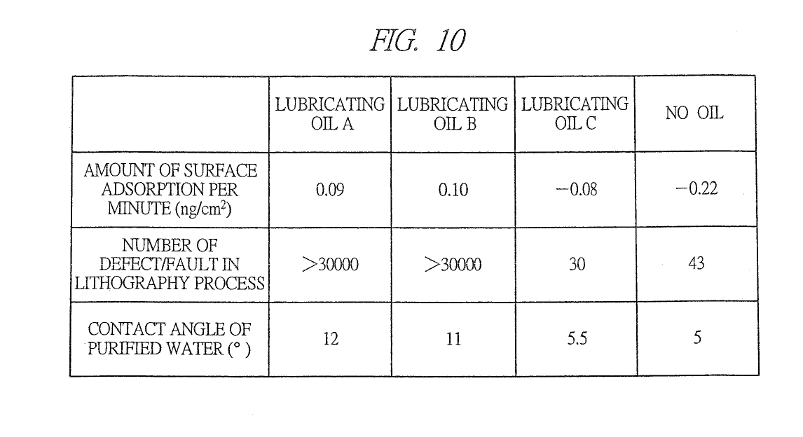

From this data, in order to obtain an average adsorption ratio of the adsorbed materials in the period concerned, the actual measurement data in FIG. 9 is further fitted so as to be linear, and a graph obtained as a result and an equation are shown. The average adsorption ratios of the respective lubricating oils are collectively shown in the second row in the table of FIG. 10.

In order to show the effectiveness of this measurement, the lubricating oil A, the lubricating oil B and the lubricating oil C were used respectively as lubricating oils to the operating mechanism parts in the CD-SEM vacuum vessel, and it was checked whether defects and faults arising from the contamination of lubricating oil components in the lithography process occurred or not, and the number of defects was measured by an apparatus using the optical scattering principle (generally sold commercially as a foreign matter inspecting apparatus). As a result, the lubricating oil A and the lubricating oil B caused defects and faults arising from the contamination of lubricating oil components in the lithography process, and the number of defects exceeded the max countable limit, so that it was described that the number of defects and faults in the lithography process was more than 30000 in the third row of the table shown in FIG. 10. On the other hand, the lubricating oil C showed the same level as that in the case where oil was not used, that is, it is the background level of the measurement apparatus, and it may be said that it did not cause defects and faults arising from the contamination of lubricating oil components in the lithography process.

In order to further confirm the above, the surface adsorption, the number of defects and faults, and the static contact angle of purified water on a surface exposed to oil (hereinafter, simply referred to as contact angle of purified) were measured. The measurement of the contact angle of purified water is used to grasp a wettability of purified water to a certain surface, that is, a change of surface energy. Its measurement principle is quite simple and it only requires just to drop purified water onto a wafer exposed to contamination in vacuum and to measure the geometric contact angle of purified water. As the apparatus to measure the contact angle of purified water, Drop Master 500 made by Kyowa Interface Science Co., Ltd., was employed. The dropping amount of the purified water for one time is about 1 μl, and an image can be obtained after 2 seconds after dropping. The result is shown in the fourth row of the table shown in FIG. 10. In the case of the lubricating oil C, the measurement result of the contact angle of purified water was 5.5° which is almost same as 5° in the case without oil within the margin of error. On the contrary, in the cases of the lubricating oils A and B, the contact angle of purified water was 12°, and so the contact angle of purified water was twice of that in the case without oil.

Generally, as it is known that fluorine coating has a water repellant effect, when fluorinated lubricant is adsorbed to the surface, the contact angle of purified water increases. The contact angle of purified water in the case of the lubricating oil C which was thought to cause a small number of defects and faults in the lithography process and not cause the contamination of lubricating oil components is almost same as that in the case without oil, and it is thought that there is no contamination on the surface due to the fluorinated lubricant. On the other hand, in the cases of the lubricating oils A and B, the contact angle of purified water increases over twice in comparison with the case without oil, therefore it is thought that the fluorinated lubrican is attached to the surface and the surface is contaminated.

According to the foregoing, the lubricating oil whose adsorption amount per minute is less than 0.09 ng/cm² after the start of vacuum evacuation and after reaching the quasi-equilibrium state in the observation by the crystal oscillator by use of the apparatus for evaluating a lubricant for performing a measurement evaluation of vaporized/adsorbed components of the lubricating oil shown in FIG. 3 shows preferable results. Also as to grease in which such oil is used as its base oil, since the vapor pressure is regulated by the base oil, the present invention is also effective to grease.

In this manner, by using lubricating oil and grease classified by the adsorption amount per unit time observed by the crystal oscillator by use of the apparatus for evaluating a lubricant for performing a measurement evaluation of vaporized/adsorbed components of lubricating oil shown in FIG. 3 to the mechanism using lubricant in the vacuum vessel in the CD-SEM shown in FIG. 1, it is possible not to cause defects and faults to samples due to the contamination by lubricating oil components in the vacuum vessel. As a result, even for an apparatus where lubricating oil and grease are applied, it is possible to control the occurrence of defects and faults including contamination to samples in the process after introducing it into a vacuum vessel.

In the foregoing, the invention made by the inventors of the present invention has been concretely described based on the embodiments. However, it is needless to say that the present invention is not limited to the foregoing embodiments and various modifications and alterations can be made within the scope of the present invention.

For example, the present invention is applicable to, not merely a CD-SEM, but also widely to general apparatuses which have mechanisms using lubricants such as oil, grease and the like in a vacuum vessel thereof.

The apparatus having a vacuum vessel according to the present invention is applicable to vacuum apparatuses such as semiconductor manufacture apparatuses having vacuum vessels and general apparatuses having mechanisms using lubricants in vacuum vessels thereof.

What is claimed is:

1. An apparatus for evaluating lubricant, comprising:
a vacuum vessel;
a quadrupole analyzer;
an angle valve connected to a surface that faces a surface connected to the quadrupole analyzer of the vacuum vessel;
a turbo-molecular pump connected to the angle valve;
a dry scroll pump connected to the turbo-molecular pump;
a plate for lubricant application provided inside the vacuum vessel; and
a crystal oscillator element provided inside the vacuum vessel.

2. The apparatus for evaluating lubricant according to claim 1,
wherein the crystal oscillator element is attached inside a flange coupled to a surface that is different from the surface to which the quadrupole analyzer of the vacuum vessel and the angle valve are connected.

3. The apparatus for evaluating lubricant according to claim 1,
wherein the plate for lubricant application has a folded structure in a U-shape having fold-back sides having different lengths.

4. The apparatus for evaluating lubricant according to claim 3,
wherein a circular hole is provided to a longer side of the fold-back sides of the plate or lubricant application.

5. The apparatus for evaluating lubricant according to claim 1,
wherein the plate for lubricant application is formed by folding a stainless plate having a length of 31 cm, a width of 16 cm, and a thickness of 1.5 mm.
